(19) United States
(12) Patent Application Publication
(10) Pub. No.: US 2006/0241970 A1
(43) Pub. Date: Oct. 26, 2006

(54) Process and device for health orientation

(76) Inventor: Aleksander Winiarski, Breman (DE)

Correspondence Address: PENDORF & CUTLIFF, 5111 Memorial Highway, Tampa, FL, 33634-7356, US

(21) Appl. No.: 11/111,225

(22) Filed: Apr. 21, 2005

(51) Int. Cl. G06Q 10/00; G06F 19/00; G06Q 50/00; A61B 5/00

(52) U.S. Cl. 705/2; 705/3

(57) ABSTRACT

The invention concerns a process for health orientation of a person comprising (a) measuring at least one body property (physical attribute or vital statistic) of the person, which characterizes a health condition of the person, with at least one measuring device (1, 3, 5, 7); (b) transmitting the at least one measured body property to an evaluation computer (15), which is connected with the at least one measuring device (1, 3, 5, 7); (c) determining, via the evaluation computer (15), whether the deviation between the at least one body property and a predetermined comparison value exceeds a set threshold; and (d) displaying an illness or abnormality information via a display means (13), in particular by a monitor, if it has been determined in step (c) that the threshold value has been exceeded. Besides this, the invention concerns a device (2) with which the process for health orientation of a person can be carried out.

### BACKGROUND OF THE INVENTION

1. Field of the Invention

The invention concerns a process and a device for the health orientation of a person. Besides this, the invention concerns a computer program which in practice in conjunction with the device carries out the process for health orientation of the person.

2. Related Art of the Invention

The treatment of illnesses is frequently associated with high costs, which in the case of serious illnesses, such as for example cancer or depression, often does not result in healing. Besides this, illnesses frequently trigger follow-up costs, such as rehabilitation or home care costs. Thus, preventive means are of immense importance in order to increase the chances of healing already afflicted persons and in order to reduce the costs associated with illnesses. This applies in particular to the industrial countries in which the costs associated with the health care system continuously strongly increase due to the increasing component of older members of the population.

The term prevention includes all measures which prevent, reduce or delay the possibility of an onset in deterioration of health. One important human-related prevention measure is that the respective persons orient themselves with regard to health, that is, that the persons inform themselves regarding their own health condition, health risk, health improving behaviors, etc.

Measures for health orientation of a person, such as for example medical examination or information dialogue, are relatively seldom carried out, since as a rule, due to the employed technical personnel (usually doctors), are associated with high cost. Besides this, the measures carried out by a doctor are associated with high expenditure of time and are inconvenient, since first a date must be scheduled, at which both doctor and patient have time, whereupon the patient must present himself to the practice of the doctor, where he usually has to wait a certain amount of time until, for example, an examination and/or an information dialogue can take place.

A person could self-monitor with commercially available measuring devices, such as blood pressure and pulse measuring devices, certain vital statistics which characterize the condition of the health of the person; however, the user of this type of measurement device does not receive a health orientation with at least a first diagnosis and with information in certain cases for further treatment or with a recommendation for a change in behavior. Thus, these measurement devices rarely contribute to prevention.

### SUMMARY OF THE INVENTION

It is the task of the invention to provide a process and a device for the health orientation of a person and to provide a computer program which enables a user to examine his state of health and to provide at least an initial diagnosis, so that illnesses can be avoided or, as the case may be, recognized early.

This task is solved by a process for health orientation of a person with the following steps:

measuring at least one body property (physical attribute or vital statistic) of the person, which characterizes the state of health of the person, with at least one measuring device,

transmitting the at least one measured body property to an evaluation computer, which is connected with the at least one measuring device,

determining, via the evaluation computer, whether the deviation between the at least one body property and a predetermined comparison value exceeds a set threshold,

displaying illness information by a display means, in particular a monitor, if it has been determined in step c) that the threshold value has been exceeded.

It can be arranged that the display means invites the person to input personal data, in particular the age and the sex of the person, and/or personal behavior patterns, which could respectively influence the health condition of the person, and that personal data and/or behavior patterns input by the person with the aid of input means are recorded in memory.

Besides this, it can be provided that the comparison value and/or the threshold value are dependent upon the recorded personal data, in particular dependent upon the sex and the age of the person.

Further, it can be provided that the at least one vital statistic is the blood pressure and/or pulse of the person.

It is further advantageous to provide the at least one physical attribute is the hearing of the person.

It is in useful manner provided, that the at least one physical attribute is vision of the person.

Besides this, in step a) the weight of the person can be determined.

Further, in step a) the height of the person can be measured.

It can be provided that each body property is associated with or assigned to a risk group comprised of various risk characteristics, which respectively could influence the condition of health of the person, and that in step a) those body characteristics are measured, of which the associated risk characteristic corresponds with the recorded, personal data and behavior pattern.

In useful manner, the illness information includes a recommendation, to consult a doctor.

Further, the illness information can include a recommendation to measure a further specific physical attribute or vital statistic.

It is preferably provided that the body properties measured in step a) are combined into a combined measurement value, in particular that the body weight is divided by the square of the body length, and that in step c) it is supplementally determined whether the combined measured value deviates from a comparison value associated with the respective combination by a predetermined threshold value associated with the respective combination.

The task is, besides this, solved by a device for health orientation of a person with

at least one measurement device for measuring at least one body property (physical attribute or vital statistic) of the person, which characterizes a health condition of the person,

an evaluation computer, which is connected with the at least one measurement device, for determining, whether the deviation of the at least one measured body property exceeds a predetermined comparison value by a predetermined threshold value,

a display means, in particular a monitor, for displaying illness information, when it has been determined that a threshold value has been exceeded, and

a control computer for controlling the at least one measurement device, the evaluation computer and the display means according to the steps of the inventive process.

It can be provided, that the device is so designed, that via the display means a request is displayed for input of personal data, in particular for input of the age and the sex of the person, and/or personal behavior characteristics, which could respectively influence the health condition of the person, and that the personal data and/or behavior characteristics input with the aid of input means by the person are recorded.

Preferably the device is so designed, that the comparison value and/or the threshold value are dependent upon the recorded personal data, in particular upon the sex and the age of the person.

Besides this, it can be provided that the device includes a device for determining the blood pressure and/or pulse.

Preferably the device includes a hearing test device for determining a measurement value characterizing the acuteness of hearing of the person.

Besides this, the device preferably includes a vision test device for determining a measurement value characterizing the visual acuity of the person.

Further, the device can include a scale for weighing the body weight of the person.

Preferably the device includes a height measuring device for measuring the length of the body of the person.

It can be provided, that the device is so adapted, that each body property is associated with at least one risk group comprised respectively of various risk characteristics which can influence the condition of health of the person, and that in step a) the respective body properties are measured, of which the associated risk characteristics correspond with the recorded, personal data and behavior patterns.

Besides this, it can be provided that the device is so adapted, that the illness information includes a recommendation to consult doctor.

Further, the device can be so adapted, that the health information includes a recommendation to measure a particular further body property.

Preferably, the device is so designed, that body properties (physical attributes or vital statistics) measured in step a) are combined into a combined measurement value, in particular that the body weight is divided by the square of the body length, and that in step c) it is further determined whether the deviation of this combination value from a predetermined comparison value associated with the respective combination exceeds a predetermined threshold associated with the respective combination.

It can be provided that the device includes a booth within which the at least one measurement device, the evaluation computer, the display means, the control computer and at least one standing or seating place for at least one person are provided.

One wall of the booth can include information means, in particular information display screens, leaflets and posters, for informing regarding in particular health relevant topics.

Therein, it can be provided that the wall is an exterior wall.

Besides this, on the booth acoustic and/or optical information means, in particular a loud speaker and/or an information display screen, can be provided for informing, in particular regarding health relevant topics.

Preferably the booth has a base or footprint which is essentially circular.

Preferably the booth includes an acoustic source, in particular a music system or a fountain, of which the sound level increases as the acoustic level penetrating into the booth from outside increases.

It can be provided that in the booth, a light source is provided for emitting light radiation of predetermined wavelengths, which is so adapted, that the intensity of the light radiation is greater than the intensity of the light penetrating the booth from outside.

Preferably an aromatic source is provided in the booth, in particular a sachet such as a lavender potpourri, of which the level of aroma is greater than the ambient level of aroma.

Preferably the booth includes a booth part enclosed essentially on all sides, in which at least the control computer and the evaluation computer are provided.

Finally, the task is solved by a computer program for controlling at least the measuring device, an evaluation computer and a display means of an inventive device according to the steps of the inventive process.

### BRIEF DESCRIPTION OF THE DRAWINGS

The invention will be described in greater detail in the following on the basis of the figures. There is shown in:

FIG. 1 a schematic diagram showing several measuring devices, an evaluation computer, a control computer, an input means and a display means of an inventive device for health orientation of the person,

FIG. 2 a perspective view of a booth of the inventive device,

FIG. 3 a top view upon the booth, and

FIG. 4 a flow diagram of the inventive process for the health orientation of the person.

### DETAILED DESCRIPTION OF THE INVENTION

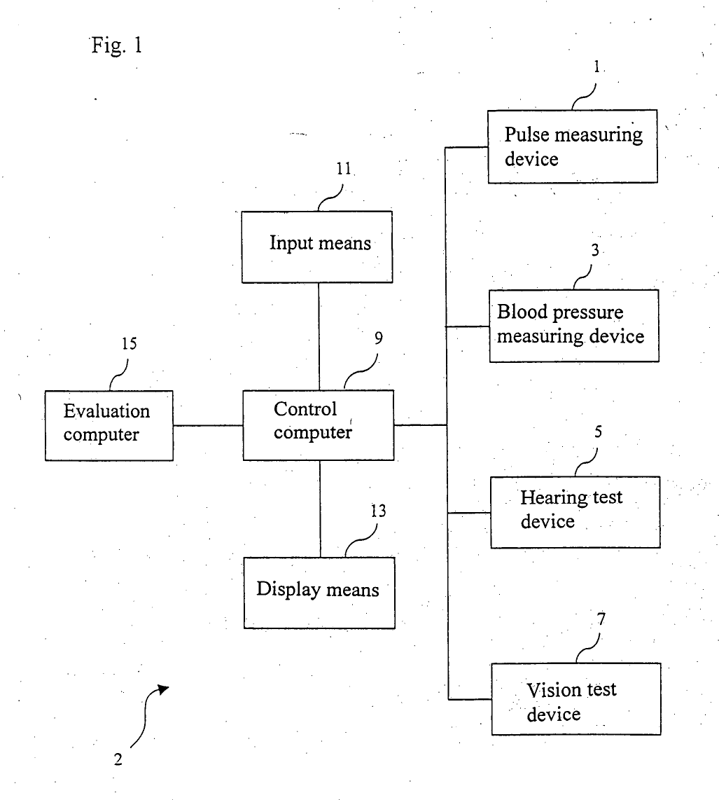

In FIG. 1, multiple measuring devices (1, 3, 5, 7) of an embodiment of the device 2 for health orientation of a person are schematically shown. These measuring devices measure the body properties characterizing the health condition of the person and in this embodiment include a pulse measuring device 1, a blood pressure measuring device 3, a hearing testing device 5 and a vision testing device 7. Alternatively, the device 2 could include fewer, more, or alternative measuring devices. For example, besides these, the weight of the user could be automatically determined with a suitable weighing device, for example with a scale integrated in the chair, or also the body height could be measured with a suitable length measuring device. Further, the self diagnosing device could include known measuring devices for measuring the heart frequency or the blood pressure.

Each measuring device is connected with a control computer 9, which controls the measuring devices 1, 3, 5, 7, via a serial or parallel data port. The control computer includes input means 11, for example a keyboard and/or a mouse, so that data can be input into the device. Besides this, the control computer 9 includes a monitor as display means 13.

Further, the device 2 includes an evaluation computer 15, which is connected with a control computer 9 and therewith also with the measuring devices 1, 3, 5, 7. The evaluation computer 15 evaluates the body properties measured by the measuring devices 1, 3, 5, 7, for example in that the measurement results are compared with the predetermined or preset comparison values of a healthy person.

The pulse measuring device 1 can include for example a cuff, which is positioned around the arm of the person, so that in known manner, the pulse can be measured. Further, the blood pressure measuring device 3 can include an automatically inflatable cuff, so that in known manner, the blood pressure of the person can be automatically measured, when the cuff is positioned about the arm of the person. Alternatively, in place of a pulse measuring device 1 and a blood pressure measuring device 3, a combined pulse-pressure measuring device be provided with one arm cuff, with which both the pulse as well as the blood pressure can be automatically measured.

The hearing testing device 5 includes a sound producer, with which sounds of various intensity and various frequencies can be produced, for example sounds can be produced digitally by a hear test computer, which could be connected to the control computer 9. Besides this, the hearing testing device 5 includes headphones, via which the person can hear the produced sounds. In order to carry out a hearing test, the hearing test device can produce various sounds of various frequencies and various sound intensities, wherein the user is tasked respectively with the aid of the display means 13 to input via the input means 11 into the control computer 9 whether a certain sound has been heard or not. In this manner, the audible frequency range and the smallest audible audio level hearable by the user can be determined.

The hearing test device can also be integrated in the control computer 9. In this case, the control computer 9 must be provided with headphones and means for producing digital sounds of various frequency and audio intensity.

The vision testing device 7 can, for example, include a housing, in which on an inner wall of the housing, for example with a projector, symbols, such as letters or numbers of various size, can be projected. The housing includes two eye openings, which are so arranged, that the user can look through these eye openings to see the symbols, wherein a frame is provided in front of each eye opening for a lens. The vision testing device includes multiple lenses, which variously influence the light beams in part. The frame is so constructed, that it can automatically exchange one lens in front of its eye opening for another lens, when it obtains an appropriate signal from the vision test computer of the vision test device, which is connected with the control computer 9. The user, who looks through the eye openings, is prompted, for example by the production of a sound signal or by an indication on the display means 13, to input via the input means 11 into the control computer 9 or via input means of the vision computer, whether he can see better with the present lens provided before the eye opening than with the lens provided there immediately previously. In this manner, various lenses are compared with each other, so that finally the lens which enables the user to see optimally can be found. The characteristics which describe this optimal lens, such as for example the diopter, can be relayed via the vision test computer to the control computer 9 and finally to the evaluation computer 15.

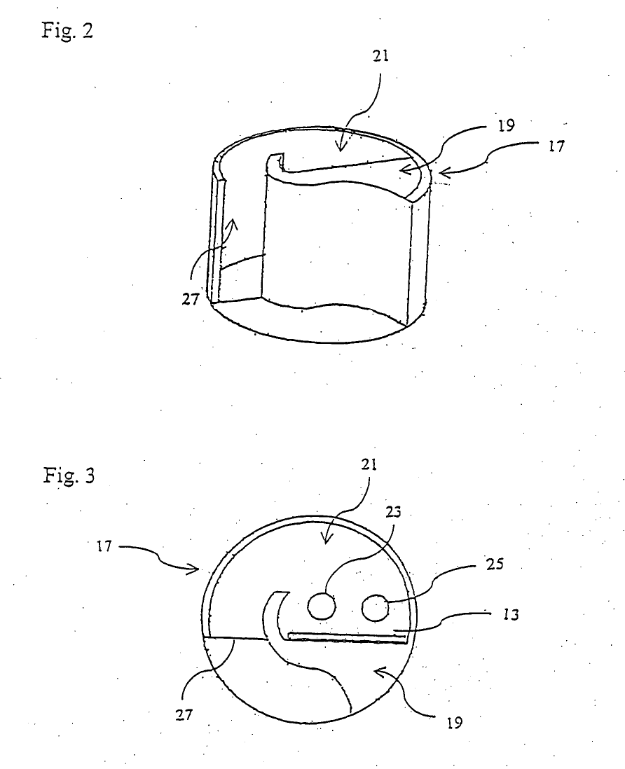

FIG. 2 shows a booth 17 of the device 2, in which the computer 9, 15, measuring devices 1, 3, 5, 7, input 11 and display means 13 shown in FIG. 1 are incorporated. The booth 17 is essentially circular shaped in its foundation or footprint (FIG. 3) and includes a first 19 and a second 21 booth part. The first booth part 19 is essentially closed on all sides, so that the control computer 9 and the evaluation computer 15, which are housed in this partial booth 19, are not visible from outside. Besides this, parts of the measuring devices, for example the vision test computer or the hearing test computer, can be incorporated in the booth part 19. The second booth part 21 borders on the first booth part 19 and is open upwards. In this partial booth 21, two seating places 23, 25 are provided for two persons in front of the display means 13. Besides this, there is located in the second booth part 21 at least the part of the measuring devices, which must be accessible for the users for carrying out the measurements, such as for example headphones for the hearing test. The second booth part 21 includes a passage 27, through which the user can enter the second booth part 21. This passage 27 can be provided with a door.

On the outer walls of the booth 17 are provided information means (not shown), such as information display screens, posters or leaflets. This information means includes information regarding health-relevant topics, in particular regarding various illnesses and health endangering behaviors, and contribute thereby to health orientation and therewith to prevention.

Further, information means for health relevant topics for health orientation can be provided on the booth 17. For example, information display screens, leaflet holders or loudspeakers with connections or other known types can be secured to the booth 17.

Besides this, audio sources, a light source and a source of aroma can be provided in the booth 17.

The sound source can, for example, be a music system or a fountain, which can be so adapted, that sounds penetrating the second booth part 21 from outside can be overshadowed by predetermined sounds, such as for example promoting the comfort or well being of the user, such as the splashing of water or calming music. This means, the sound intensity of the sound source should be increased along with the audio intensity of the sounds penetrating the booth part 21.

The light source can be a known light source, which emits a predetermined spectrum of wavelengths and is so adapted, that light penetrating the booth part 21 from outside is superimposed at least in the area of the booth part 21, in which the seat places 23, 25 are located. The light sources can be for example blue light emitting, in order to calm a person sitting on the seat. The intensity of the light emitted by the light source should be greater than the intensity of the light penetrating the booth part 21 from the outside.

As aroma source there can be provided for example a lavender sachet or potpourri, which is provided in the booth part 21 in the area of the seats 23, 25. Also, the intensity of the aroma source, that is for example the amount of lavender fragrance, is to be so controlled, that the aromas pre-existing in the booth part 21 are covered over by the aroma of the aroma source.

The booth 17 can be produced from any suitable material, for example wood, plastic, glass and/or aluminum.

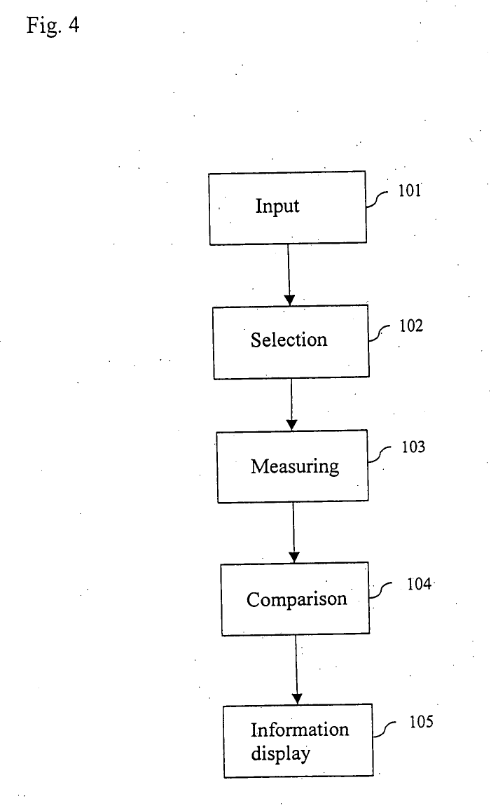

FIG. 4 shows the sequence of an inventive process for health orientation of a person, which can be carried out with the device according to FIG. 1.

In step 101 a person, who is seating on one of the seats 23, 25 in front of the display means 13 and the booth part 21, is asked by the display means 13, to input into the control computer 9, with the aide of the input means 11, personal data which could influence his health condition, such as age and sex, or personal behaviors which likewise could influence his health condition, such as smoker/non-smoker. The personal data and behavior characteristics input by the user are recorded in the control computer 9 or in the evaluation computer 15.

In step 102 at least one of the measuring devices 1, 3, 5, 7 is selected, with which a body property is to be measured. This selection can be carried out in that with the aide of the display means 13 the user is asked to select via the input means 11 at least one of the measuring devices 1, 3, 5, 7. The inputs of the user can then be recorded in the control computer 9 or in the evaluation computer 15.

Alternatively, the control computer 9 or the evaluation computer 15 can automatically select a measuring device 1, 3, 5, 7. In addition to this, in one of the computers 9, 15, various risk groups can be recorded, which respectively are associated with the body properties measurable with the self diagnostic devices. Each risk group is characterized by various risk characteristics, wherein risk characteristics are characterized thereby, that persons, who exhibit these characteristics, have a greater likelihood of suffering from an illness associated body condition or, as the case may be, would suffer, than persons who do not exhibit these characteristics. Risk characteristics are for example smoking, stress, alcoholism, frequent fatigue, etc. These risk characteristics are compared with the personal data and behavior patterns recorded in step 101 by the evaluation computer 15 or by the control computer 9, wherein the measuring devices 1, 3, 5, 7 or, as the case may be, the body properties are selected of which the associated risk groups correspond with the input personal data and behavior patterns, in particular within a range of tolerance.

In step 103 the selected measuring devices are used to measure the corresponding body properties in known manner, as already described above by way of example.

The measured body properties are transmitted to the evaluation computer 15 via the control computer 9. In other embodiments, the measured body properties can also be transmitted directly from the measuring devices 1, 3, 5, 7 to the evaluation computer 15. In that case, the measuring devices 1, 3, 5, 7 must, however, be directly connected with the evaluation computer 15.

The evaluation computer 15 determines in step 104 whether the deviation of a measured value from the comparison value recorded in the evaluation computer 15 is greater than a predetermined threshold value, in certain cases recorded in the evaluation computer 15. Therein the comparison value and the threshold value depend upon the respective measuring devices and additional respective body property influencing personal data and/or behaviors, which were recorded in step 101 in the evaluation computer 15 or the control computer 9. The respective comparison value can be the same value as that which was measured for a healthy person with the corresponding measuring device, which healthy person has the same personal data, in particular the same age and the same sex, as the user of the device 2. For a 30 year old man, the pulse measuring device 1 could have a comparison value of, for example, 70 beats per minute. The respective threshold value can be the value of the maximal deviation of a measured body property of a user from the respective comparison value which, in accordance with the standards of medicine, represents a still healthy value. Therein, for each comparison value, two threshold values can be provided; a first threshold, which contemplates a deviation in the direction towards smaller measured values, and a second threshold value, which takes into consideration a deviation in the direction towards greater measured values. In the above example, for the pulse rate, a first threshold value could be 15 beats per minute, and the second threshold value 30 beats per minute, so that a pulse between 55 and 100 beats per minute is not evaluated as unhealthy for a 30 year old man.

Alternatively, multiple first and second threshold values can be allocated for each comparison value, so that with the self diagnosis device, the seriousness of an illness condition can be assessed. The illness condition is considered the most serious when the greatest threshold value is exceeded, in comparison to which, when only the smallest threshold is exceeded, the deviation from the comparison value is relatively small, so that the health condition is evaluated as only slightly abnormal.

Further, in step 104, a combination measured value could be determined from the various measured body properties in a predetermined manner of computation, that is, by a predetermined combination of measured values. Besides the measuring devices, then each type of measurement would be associated with at least one comparison value and at least one threshold, which are likewise recorded in control computer 9 and evaluation computer 15 and which could depend from the personal data, the sex and the age. For example, if the weight and the body height of the user had been measured in step 103, the so called body-mass-index (BMI) can be determined, in that the body weight in kilogram is divided by the square of the body height in meters. For a 30 year old man, the comparison value could be 21.7 kg/m² and the threshold 3.2 kg/m².

In step 105 an illness or abnormality information is displayed on display screen 13, here a monitor, if in step 104 an exceeding of a threshold has been determined. For this, a variety of illness or abnormality information is recorded in control computer 9 or in evaluation computer 15, wherein each measuring device is associated with at least one illness or abnormality information. The illness information contains the results of the comparison, that is, the information that the measured body property could indicate an illness. Besides this, the illness information can include a recommendation to consult a doctor, in particular a specialist, and tips for appropriate behavior which could lead to an improvement of the health condition. Further, when an exceeding of a threshold value results from a measurement with a hearing test device 5 or a visual test device 7, a visit to a sales location for hearing aids or an optometrist could be recommended.

Besides this, the illness or abnormality information could include a recommendation to carry out a particular further measurement of a body property with the device 2. For example, if it is known that when a deviation of one measured body property from the appropriate comparison value exceeds a threshold, frequently also another body property corresponding deviation is exhibited, of which the illness or abnormality information could include a recommendation precisely to check this body property with the device 2.

If multiple first and second threshold values are associated in step 104 for one comparison value, then for each of these threshold values restrictively, one illness or abnormality information is recorded in the control computer 9 or evaluation computer 15. For example, the illness information that if only the smallest threshold value is exceeded, could be to recommend a healthy orientation by measuring again the body properties with the device 2 after a predetermined time interval, for example, after several days, or to repeat at a different time of day, without immediately consulting a specialist. If in contrast a greater threshold is exceeded, then the illness information can indicate a recommendation to immediately consult a doctor.

If it was determined in step 104 that no threshold value was exceeded, then the display means 13 can indicate health information. Each measuring device is associated with at least one health information, which is recorded respectively in the control computer 9 or evaluation computer 15. If a measuring device is assigned multiple health information or messages, then these can be associated with the various personal data, such as age or sex. Then on the display means 13 respectively that health information is displayed which is associated with the respective measuring device and the respective personal data. The health information can provide, in addition to information that the measured body properties do not indicate any illness or abnormality, recommendations for behaviors which maintain a positive condition of health.

If combination measured values are determined in step 104 from various measured body properties, then in addition, for each type of calculation of a combined measure value, respectively, at least one illness information and one health information is recorded in control computer 9 or evaluation computer 15. If one deviation of one of the combination measured values exceeds the corresponding threshold value of the corresponding comparison value, then the respective type of calculation associated illness information is displayed on display means 13. Otherwise, a health message is displayed. Also, this illness and health information can respectively depend upon personal data such as sex and age.

Multiple body properties measurable with the device 2 can also be associated with one illness information, wherein the respective illness information is displayed if all associated body properties are measured and for each of these body properties, the deviation from the respective corresponding comparison value is greater than the respective corresponding threshold value. This illness information can, for example, indicate that the determined deviation from the comparison values indicates a certain illness.

The characteristics disclosed in the above description, the claims as well as the attached drawings, can individually, or in combination, be modified for accomplishment of the invention in its diverse embodiments.

### REFERENCE NUMBER LIST

1 Pulse measuring device
2 Device for health orientation
3 Blood pressure measuring device
5 Hearing test device
7 Vision test device
9 Control computer
11 Input means
13 Display means
15 Evaluation computer
17 Booth
19 First booth part
21 Second booth part
23, 25 Seats
27 Passage

1-35. (canceled)

36. A process for health orientation of a person comprising the following steps:
a) measuring with at least one measuring device (1, 3, 5, 7) at least one body property of the person which characterizes a health condition of the person;
b) transmitting the at least one measured body property to an evaluation computer (15), which is connected with the at least one measuring device (1, 3, 5, 7);
c) determining, via the evaluation computer (15), whether the deviation between the at least one body property and a predetermined comparison value exceeds a set threshold; and
d) displaying illness or abnormality information by a display means (13), in particular a monitor, if it has been determined in step c) that the threshold value has been exceeded.

37. The process according to claim 36, wherein the display means (13) displays a request for input of personal physical data and/or personal behaviors, which could respectively influence the condition of health of the person, and wherein the personal physical data and/or behaviors input by the person with the aide of input means (11) are recorded.

38. The process according to claim 37, wherein the comparison value and/or the threshold value are dependent upon the recorded personal data, in particular the sex and the age of the person.

39. The process according to claim 36, wherein the at least one body physical property is selected from the group consisting of the blood pressure of the person, the pulse of the person, the hearing of the person, and the vision of the person.

40. The process according to claim 36, wherein in step a) at least one of the weight and the body height of the person is measured.

41. The process according to claim 37, wherein each body property is assigned at least one risk group comprised of various risk characteristics which could respectively influence the health condition of the person, and that in step a) those respective body properties are measured of which the associated risk characteristics correspond with the recorded personal data and behaviors.

42. The process according to claim 36, wherein the illness or abnormality information includes a recommendation to consult a doctor or a recommendation to measure a particular further body property.

43. The process according to claim 36, wherein the body properties measured in step a) are integrated into a combined measured value, in particular that the body weight is divided by the square of the body height, and that in step c) it is supplementally determined whether a deviation of the combination measured value from a predetermined comparison value associated with the respective combination exceeds a preset threshold value associated with the respective combination.

44. A device for health orientation comprising:
at least one measuring device (1, 3, 5, 7) for measuring at least one body property of the person, which characterizes a health condition of the person,
an evaluation computer (15), which is connected with at least one measuring device (1, 3, 5, 7), for determining whether a deviation of at least one measured body property exceeds a predetermined comparison value by a predetermined threshold value,
a display means (13), in particular a monitor, for displaying an illness or abnormality information, in the case that it is determined that a threshold value has been exceeded, and
a control computer (9) for controlling the at least one measuring device (1, 3, 5, 7), the evaluation computer (15) and the display means (13) by the following steps: (a) measuring with at least one measuring device (1, 3, 5, 7) at least one body property of the person which characterizes a health condition of the person; (b) transmitting the at least one measured body property to an evaluation computer (15), which is connected with the at least one measuring device (1, 3, 5, 7); (c) determining, via the evaluation computer (15), whether the deviation between the at least one body property and a predetermined comparison value exceeds a set threshold; and (d) displaying illness or abnormality information by a display means (13), in particular a monitor, if it has been determined in step c) that the threshold value has been exceeded.

45. The device according to claim 44, wherein the device is so adapted that the display means (13) displays a request for input of personal data, in particular for input of the age and the sex of the person and/or personal behaviors, which respectively could influence the health condition of the person, and that personal data and/or behaviors input by the person with the aide of input means (11) are recorded.

46. The device according to claim 45, wherein the device is so adapted, that the comparison values and/or the threshold values are dependent upon the recorded personal data, in particular the sex and the age of the person.

47. The device according to claim 44, wherein the device includes at least one of a device for determining the blood pressure and/or pulse, a hearing test device (5) for determining a measurement value characterizing the hearing ability of the person, a vision test device (7) for determining a measurement value characterizing the vision of the person, a scale for weighing the body weight of the person, and a length measuring device for measuring the height of the person.

48. The device according to claim 44, wherein the device is so adapted, that each body property is associated with at least one risk group comprised of various risk characteristics, which respectively can influence the state of health of the person, and that in step a) those body properties are measured, of which the associated risk characteristics correspond with the recorded personal data and behavior.

49. The device according to claim 44, wherein the device is so designed, that the illness or abnormality information includes a recommendation to consult a doctor or to measure a particular further body property.

50. The device according to claim 44, wherein the device is so designed, that the body properties measured in step a) are integrated into a combined measurement value, in particular that the body weight is divided by the square of the body length, and that in step c) it is supplementally determined whether a deviation of a combined measurement value from a predetermined comparison value associated with the respective combination exceeds a predetermined threshold value associated with the respective combination.

51. The device according to claim 44, wherein the device includes a booth (17), in which the at least one measuring device (1, 3, 5, 7), the evaluation computer (15), the display means (13), the control computer (9), and at least one standing place or seat (23, 25) for at least one person is provided.

52. The device according to claim 51, wherein one wall of the booth (17) includes information means, in particular at least one of information display screens, leaflets and posters, for providing information regarding health relevant topics.

53. The device according to claim 52, wherein the wall is an outer wall.

54. The device according to claim 51, wherein on the booth (17) acoustic and/or optical information means, in particular a loudspeaker and/or an information display screen, are provided for providing information regarding health relevant topics.

55. The device according to claim 51, wherein the booth (17) has an essentially circular footprint or is upwards at least partially open.

56. The device according to claim 51, further comprising at least one of:
(a) a sound source, in particular a music system or a fountain, in the booth (17), of which the sound intensity is greater than the sound intensity of the sound entering the booth (17) from outside,
(b) a light source in the booth (17) for emission of light of predetermined wavelengths, which is so adapted, that the intensity of this light is greater than the intensity of the light penetrating from outside the booth, and
(c) a fragrance source, in particular a potpourri or a lavender sachet, of which the aroma intensity is greater than the aroma intensity of other existing smells.

57. The device according to claim 51, wherein the booth (17) includes a booth part (19) closed essentially on all sides, in which at least the control computer (9) and the evaluation computer (15) are provided.

58. A computer program for a control computer (9) for controlling at least one measuring device (1, 3, 5, 7), an evaluation computer (15) and a display means (13) of a device for health orientation comprising:
at least one measuring device (1, 3, 5, 7) for measuring at least one body property of the person, which characterizes a health condition of the person,
an evaluation computer (15), which is connected with at least one measuring device (1, 3, 5, 7), for determining whether a deviation of at least one measured body property exceeds a predetermined comparison value by a predetermined threshold value,
a display means (13), in particular a monitor, for displaying an illness or abnormality information, in the case that it is determined that a threshold value has been exceeded, and
a control computer (9) for controlling the at least one measuring device (1, 3, 5, 7), the evaluation computer (15) and the display means (13) by the following steps: (a) measuring with at least one measuring device (1, 3, 5, 7) at least one body property of the person which characterizes a health condition of the person; (b) transmitting the at least one measured body property to an evaluation computer (15), which is connected with the at least one measuring device (1, 3, 5, 7); (c) determining, via the evaluation computer (15), whether the deviation between the at least one body property and a predetermined comparison value exceeds a set threshold; and (d) displaying illness or abnormality information by a display means (13), in particular a monitor, if it has been determined in step c) that the threshold value has been exceeded.
